(19) United States
(12) Patent Application Publication
(10) Pub. No.: US 2023/0081105 A1
(43) Pub. Date: Mar. 16, 2023

(54) EXOSOME-DELIVERED TARGETING TREATMENT FOR BLOOD VESSELS

(71) Applicant: University of South Carolina, Columbia, SC (US)

(72) Inventors: Wenbin Tan, Columbia, SC (US); Rong Yin, Columbia, SC (US); Li Meng, Shanghai (CN)

(73) Assignee: University of South Carolina, Columbia, SC (US)

(21) Appl. No.: 17/865,513

(22) Filed: Jul. 15, 2022

Related U.S. Application Data
(60) Provisional application No. 63/244,322, filed on Sep. 15, 2021.

(51) Int. Cl. A61K 41/00; A61B 18/20; A61N 5/06; A61K 9/50

(52) U.S. Cl. CPC A61K 41/0057; A61B 18/203; A61N 5/06; A61K 9/5068; A61B 2018/00452

(57) ABSTRACT

Described herein are systems and methods for a type of paradigm-shift nanoparticles functionalized endothelial optical exosomes for vascular malformation treatment, including Port Wine Stain, using exosomes as a drug delivery vehicle in combination with Near-Infrared-mediated laser therapy.

### STATEMENT REGARDING FEDERALLY SPONSORED RESEARCH

This disclosure was made with government support under R01 AR073172 awarded by National Institute of Health and W81XWH1810096 awarded by the Department of Defense. The government may have certain rights in the disclosure.

### TECHNICAL FIELD

The subject matter disclosed herein is generally directed to systems and methods for a type of paradigm-shift nanoparticles functionalized endothelial optical exosomes (FEOE) for vascular malformation treatment, including Port Wine Stain (PWS), using exosomes as a drug delivery vehicle in combination with Near-Infrared (NIR)-mediated laser therapy.

### BACKGROUND

Vascular malformations are congenital vascular anomalies, present at birth, grow proportionally with age, and do not regress naturally. They usually result from developmental impairments of vasculatures including veins, arteries, capillaries and lymphatic vessels. Vascular malformations occur in 1.5 percent of the general population.

The treatments for these disorders are limited, which is an unmet need. Vascular malformations cause a variety of severe symptoms, depending on body location and types of vasculatures involved, which makes clinical management very challenging. The treatment options for vascular malformations are limited due to their complexity of types of blood vessels involved and severity of symptoms. Proper diagnosis and managements are difficult and prognosis varies, which is a major challenge and threat to public health in general.

For example, the Pulse Dye Laser (PDL) remains the treatment of choice for Port Wine Stain (PWS). Unfortunately, complete removal occurs in less than 10% patients treated. These inadequate clinical outcomes mainly result from: (1) incomplete ablation of PWS blood vessels located in the deep dermis where light from the PDL cannot reach effectively; and (2) the regrowth of blood vessels from post-PDL resistant PWS hDMVECs (p-hDMVECs).

Accordingly, it is an object of the present disclosure to provide a type of paradigm-shift nanoparticles FEOE for vascular malformations treatment, including PWS. The entire concept of using exosomes as the drug delivery vehicle in combination with the Near-Infrared (NIR)-mediated laser therapy is very novel.

Citation or identification of any document in this application is not an admission that such a document is available as prior art to the present disclosure.

### SUMMARY

The above objectives are accomplished according to the present disclosure by providing in one embodiment, a novel functionalized endothelial optical exosome. The exosome may include a chromophore delivery vehicle comprising at least one n-hDMVEC-derived exosome, at least one antibody or peptide affixed to the novel functionalized endothelial optical exosome configured to recognize and bind with at least one marker for at least one p-hDMVEC, and at least one therapeutic chromophore loaded onto the n-hDMVEC-derived exosome. Further, the at least one therapeutic chromophore may be selected from Verteporfin, ICG, or a photosensitizer. Still further, exposure of the novel functionalized endothelial optical exosome to near-infrared laser irradiation may destroy lesional or abnormal blood vessels in a subject.

In a further embodiment, the disclosure provides a method for making a novel functionalized endothelial optical exosome. The method may include forming a chromophore delivery vehicle comprising at least one n-hDMVEC-derived exosome, affixing at least one antibody or peptide to the novel functionalized endothelial optical exosome and configuring the at least one antibody or peptide to recognize and bind with at least one marker for at least one p-hDMVEC, and loading at least one therapeutic chromophore onto the n-hDMVEC-derived exosome. Further, the method may include selecting the at least one therapeutic chromophore from Verteporfin, ICG, or a photosensitizer.

In a still further embodiment, the disclosure provides a method for treating Port Wine Stain. The method may include delivering to a subject a novel functionalized endothelial optical exosome wherein the exosome includes a chromophore delivery vehicle comprising at least one n-hDMVEC-derived exosome, at least one antibody or peptide affixed to the novel functionalized endothelial optical exosome configured to recognize and bind with at least one marker for at least one p-hDMVEC, and at least one therapeutic chromophore loaded onto the n-hDMVEC-derived exosome, and the method may include exposing the novel functionalized endothelial optical exosome to near-infrared laser irradiation to destroy lesional or abnormal blood vessels in the subject. Still further, the method may include selecting at least one therapeutic chromophore from Verteporfin, ICG, or a photosensitizer.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows at: (a) absorption spectra of free Vtf; and (b) the standard curve of Vtf concentration versus its absorbance.

FIG. 2 shows Absorption spectra of free Vtf (50 μg/ml) and Vtf-encapsulated exosomes after being loaded in PBS buffer containing 50 μg/ml Vtf.

FIG. 3 shows at: (A) Vtf with various concentrations (12.5-200 μg/ml); and (B) Vtf (50 μg/ml) was loaded into exosomes with various concentrations (50-1,000 μg/ml).

FIG. 4 shows at: (A) Vtf signal showed no differences among GFP expression cells; and (B) flow cytometry spectrum of Vtf in ADAM30-CFP expression cells after being incubated with either FEOEs or ADAM30-FEOEs.

FIG. 5 shows fluorescent biodistribution of Vtf in skin and brain after PBS or FEOEs administration into a wild type mouse.

FIG. 6 shows photothermal destruction on dermal blood vessels with FEOEs administration into a wild type mouse, followed by an NIR radiation two hours later.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Unless defined otherwise, technical and scientific terms used herein have the same meaning as commonly understood by one of ordinary skill in the art to which this disclosure pertains.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, and cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise that induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition to the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, sub arachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “control” can refer to an alternative subject or sample used in an experiment for comparison purpose and included to minimize or distinguish the effect of variables other than an independent variable.

The term “optional” or “optionally” means that the subsequent described event, circumstance or substituent may or may not occur, and that the description includes instances where the event or circumstance occurs and instances where it does not.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

The purpose of this disclosure aims at engineering novel endothelial exosome-derived optical nanoparticles for treatment of Port wine stain (PWS), which can be ultimately developed as a personalized precision photodynamic therapy (PDT) for congenital vascular malformations (CVM) and other vascular diseases including tumor vasculatures.

In this disclosure, we develop a novel type of optical nanoparticles, which are referred to as functionalized endothelial optical exosomes (FEOE), to tackle these clinical barriers. We will use normal hDMVEC (nhDMVEC)-derived exosomes as the basic biological constructs to engineer FEOEs. These FEOEs present nhDMVEC tropisms, have relative stable structures in nano-sizes, are non-immunogenic and fuse with high efficiency to the cellular membrane. In addition, surface markers, such as CD133/CD166/EphB1/EphrinB2/ADAM30, on lesional hDMVECs provide a molecular basis to design FEOE for specific targeting.

The proposed FEOE will be comprised of three components: (1) n-hDMVEC-derived exosomes as the therapeutic chromophore delivery vehicle; (2) an antibody or peptide that can recognize any of those surface markers to be conjugated for specific targeting of p-hDMVECs; and (3) Therapeutic chromophore, such as Verteporfin (Vtf) or ICG or other photosensitizer or therapeutic agents as the to be loaded into n-hDMVECderived exosomes, which can be activated upon near infrared (NIR) laser irradiation to destroy lesional or abnormal blood vessels.

The formulation of FEOE is novel in nanomedicine, vesicle biology and light therapy since no such design has been ever reported. The engineered FEOE are very significant because they are designed to directly address the clinical limitations of current PWS treatment of PWS: (1) the NIR wavelength can penetrate deeper into human skin than pulsed dye laser (PDL) thus targeting those blood vessels in the reticular layer of the dermis; (2) exosomes as the drug delivery vehicle can efficiently transport the therapeutic agents into the targets; and (3) the chimera molecule on FEOE will guide specific targeting of p-hDMVECs. The FEOE is also of significance as a method of creating new nanop articles for broad new research applications for other types of vascular malformations with serious complications and limited treatment options, such as cerebral arteriovenous malformations and tumor vasculatures, etc.

Loading Vtf into n-hDMVECs Exosomes

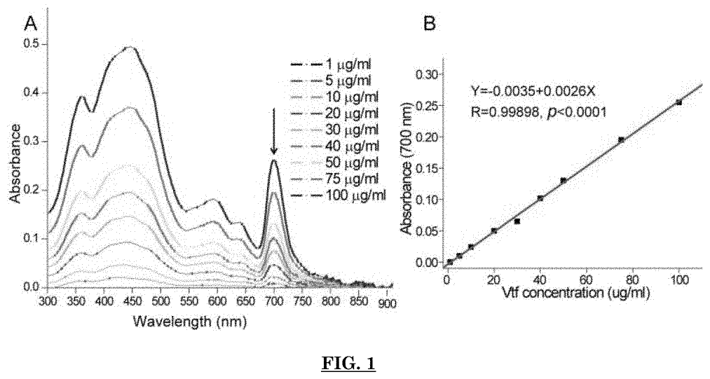

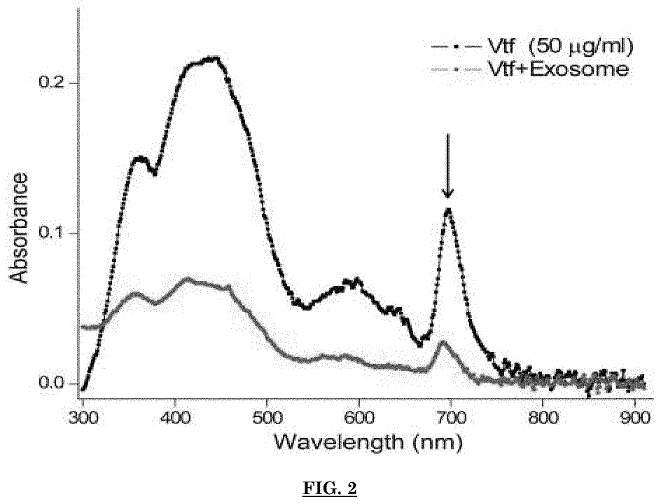

The current disclosure first characterized the absorption spectra of free Vtf. The Vtf showed a characteristic absorption peak at 700 nm (FIG. 1 at A). The peak intensities at 700 nm showed a linear correlation to the Vtf concentrations (FIG. 1 at B). We then purified n-hDMVECs derived exosomes from the conditioned culture medium using ultracentrifugation. We used a modest concentration of Vtf (50 μg/ml) in the loading buffer to evaluate the loading capacity of exosomes. Sonication was used to facilitate the loading process. The Vtf-loaded exosomes were purified using an exosome spin column kit (3K MW, ThermoFisher) to remove free Vtf. The success of Vtf loaded into exosomes was determined by the absorption spectra (FIG. 2). The concentration of loaded Vtf in exosomes was determined as 12.5 μg/ml by the peak intensity at 700 nm via the pre-determined standard curve in FIG. 1 at B. FIG. 1 shows at: (A) Absorption spectra of free Vtf (1˜100 μg/ml); The arrow indicates the peak absorbance at 700 nm; and (B) Standard curve of Vtf concentration vs its absorbance at 700 nm. FIG. 2 shows absorption spectra of free Vtf (50 μg/ml) and Vtf-encapsulated exosomes after being loaded in PBS buffer containing 50 μg/ml Vtf.

Determining Vtf Loading Efficiency.

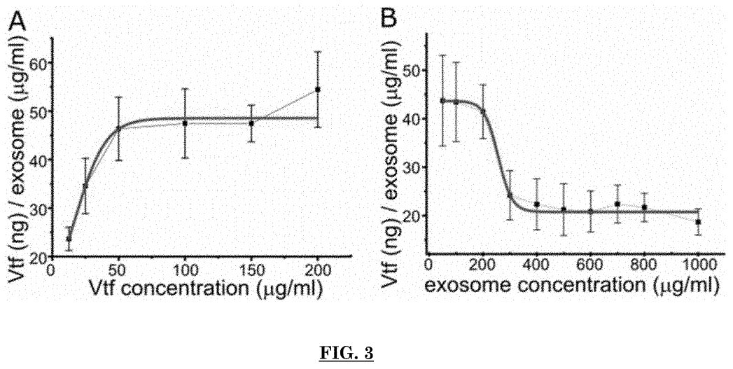

We optimized the Vtf and exosome concentrations for engineering FEOEs. Briefly, exosomes released from n-hDMVECs were collected from culture medium and purified using a gel filtration column. We then used a sonication method to load Vtf into the exosomes. Purified exosomes (˜1011 exosomes) were mixed with Vtf for sonication, followed by a size exclusion chromatography to remove the excessive free Vtf and obtain FEOEs. The concentration of loaded Vtf in exosomes was determined by the peak absorbance intensity at 700 nm via a pre-determined standard curve. We found that the highest loaded Vtf into exosomes could be achieved using 50 μg/ml Vtf to be loaded into 200 μg/ml exosomes (FIG. 3). In such an optimized condition, the Vtf loading efficiency into exosomes was estimated to be 19%. FIG. 3 shows at: (A) Vtf with various concentrations (12.5-200 μg/ml) was loaded into 200 μg/ml exosomes. The threshold concentration of Vtf reaching to the plateau was determined as 50 μg/ml and at (B) Vtf (50 μg/ml) was loaded into exosomes with various concentrations (50-1,000 μg/ml). The maximal concentration of exosomes for loading was determined as 200 μg/ml.

Engineering ADAM30-FEOEs for Specific Targeting.

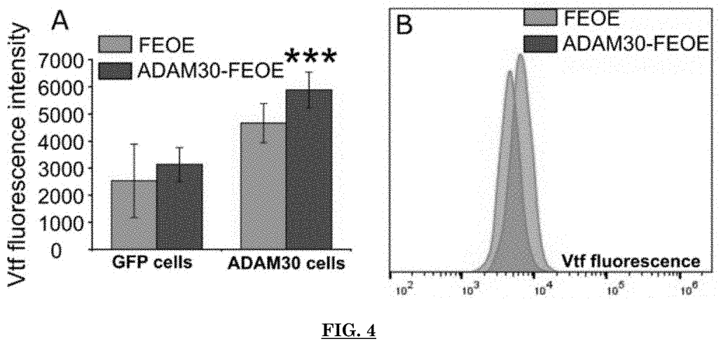

We used a crosslinker, DSPE-PEG-COOH, to insert the DSPE group into lipid layers of exosomes and attached COOH group covalently to an anti-ADAM30 antibody (FIG. 4). This antibody could recognize the extracellular domain of ADAM30. The Vtf was loaded this exosome to fabricate ADAM30-FEOEs. In order to prove the principle, we transfected human kidney HEK293 cells either with an ADAM30-cyan fluorescent protein (CFP) lentiviral vector or green fluorescent protein (GFP) control lentiviral vector. In such a situation, HEK293 cells could either over-express ADAM30-CFP fusion protein or GFP which was a control. Three days later, Vtf-loaded exosome (FEOEs) or ADAM30-FEOEs (equivalent to 250 ng of Vtf in 500 μl medium) were added into the cells and incubated for 1 hr and half. The cells then were trypsinized and analyzed by flow cytometry. The ADAM⁺ cells (CFP, excitation: 440 nm; emission: 480 nm) and GFP⁺ cells (excitation: 390 nm; emission: 509 nm) were sorted. The Vtf fluorescent signal (excitation: 420 nm; emission: 700 nm) in each type of cells were analyzed. After being incubating the cells with FEOEs or ADAM30-FEOEs, there was no difference of the average Vtf fluorescent signal per cell in the GFP expression cells; while ADAM30-FEOEs delivered significantly more Vtf into ADAM30-expression cells than FEOEs (n=4 independent experiments, FIG. 4), demonstrating that an antibody conjugation of FEOEs could facilitate a specific delivery into targeting cells. FIG. 4 shows Human kidney HEK293 cells were transfected with ADAM30-CFP or GFP control lentiviral vector. The cells then were incubated with FEOEs or ADAM30-FEOEs. A flow cytometry was used to analyze Vtf signal in each GFP or ADAM30-CFP expression cells. FIG. 4 shows at: (A) Vtf signal showed no differences among GFP expression cells; while ADAM30-FEOEs delivered significantly more Vtf in ADAM30-CFP expression cells than FEOEs. N=4; ***P<0.05 and at (B) Flow cytometry spectrum of Vtf in ADAM30-CFP expression cells after being incubated with either FEOEs or ADAM30-FEOEs.

Determining the Biodistribution of FEOEs In Vivo

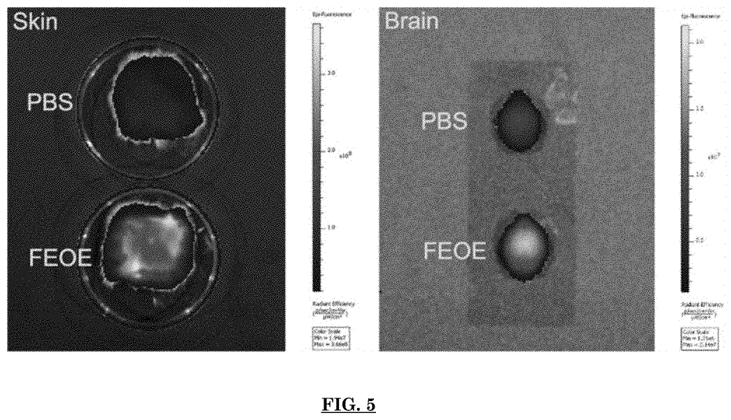

We delivered FEOEs or PBS into wild type mice (˜4 week old) via retro-orbital injections. Two hours later, we scarified animals, removed the brain and skull skin, and monitored under the IVIS-FB imaging system. The tissue biodistribution of Vtf in brain and skin was successfully analyzed (FIG. 5). FIG. 5 shows fluorescent biodistribution of Vtf in skin and brain after PBS or FEOEs administration into a wild type mouse. The tissue images were acquired using an IVIS-FB.

FEOE-Delivered Photothermal Destruction of Dermal Blood Vessels In Vivo

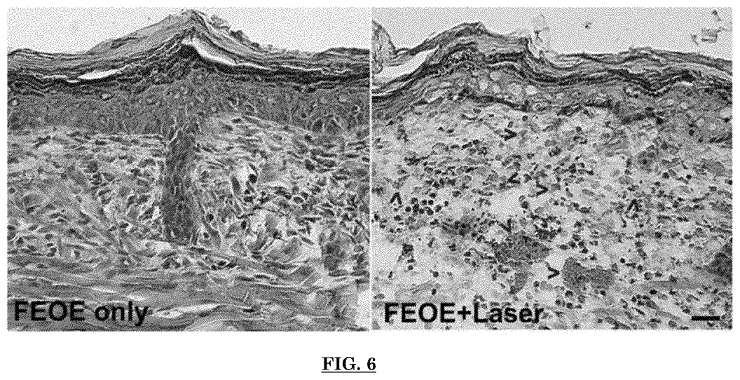

We delivered FEOEs into wild type pups (P0-1) via facial vein injections. Two hours later, a NIR (700 nm) light was given to one side of facial skin at 100 mW/cm² for 5 mins. Two days later, we scarified the animals, removed the facial skins, and checked the photothermal destruction of dermal blood vessels. We found that numerous coagulated and scatted red blood cells in dermis that were photothermal injured after an NIR radiation, while contralateral skin without an NIR exposure appeared to be normal (FIG. 6). Our results demonstrated that FEOEs delivered Vtf could render a blood vessel injury upon light radiation in vivo. FIG. 6 shows photothermal destruction on dermal blood vessels with FEOEs administration into a wild type mouse, followed by an NIR radiation two hours later. Animals were sacrificed two days later. The contralateral skin without NIR radiation appeared to have normal dermis. The light treatment side of facial dermis showed numerous coagulated and scatted red blood cells that had been photothermal agglutinated (H&E staining).

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A novel functionalized endothelial optical exosome comprising:
a chromophore delivery vehicle comprising at least one n-hDMVEC-derived exosome;
at least one antibody or peptide affixed to the novel functionalized endothelial optical exosome configured to recognize and bind with at least one marker for at least one p-hDMVEC; and
at least one therapeutic chromophore loaded onto the n-hDMVEC-derived exosome.

2. The novel functionalized endothelial optical exosome of claim 1, wherein the at least one therapeutic chromophore is selected from Verteporfin, ICG, or a photosensitizer.

3. The novel functionalized endothelial optical exosome of claim 1, wherein exposure of the novel functionalized endothelial optical exosome to near-infrared laser irradiation destroys lesional or abnormal blood vessels in a subject.

4. A method for making a novel functionalized endothelial optical exosome comprising:
forming a chromophore delivery vehicle comprising at least one n-hDMVEC-derived exosome;
affixing at least one antibody or peptide to the novel functionalized endothelial optical exosome and configuring the at least one antibody or peptide to recognize and bind with at least one marker for at least one p-hDMVEC; and
loading at least one therapeutic chromophore onto the n-hDMVEC-derived exosome.

5. The method for making a novel functionalized endothelial optical exosome of claim 4, further comprising selecting the at least one therapeutic chromophore from Verteporfin, ICG, or a photosensitizer.

6. A method for treating Port Wine Stain comprising:
delivering to a subject a novel functionalized endothelial optical exosome comprising:
a chromophore delivery vehicle comprising at least one n-hDMVEC-derived exosome;
at least one antibody or peptide affixed to the novel functionalized endothelial optical exosome configured to recognize and bind with at least one marker for at least one p-hDMVEC; and
at least one therapeutic chromophore loaded onto the n-hDMVEC-derived exosome
exposing the novel functionalized endothelial optical exosome to near-infrared laser irradiation to destroy lesional or abnormal blood vessels in the subject.

7. The method for treating Port Wine Stain of claim 6, further comprising selecting at least one therapeutic chromophore from Verteporfin, ICG, or a photosensitizer.
